(19) United States
(12) Patent Application Publication
(10) Pub. No.: US 2025/0205555 A1
(43) Pub. Date: Jun. 26, 2025

(54) COMPUTERIZED ROUTE BUILDING SYSTEM AND METHOD

(71) Applicant: PAVÉ ROUTES, LLC, Canton, OH (US)

(72) Inventor: Sebastian S. Hendricks, Canton, OH (US)

(21) Appl. No.: 19/065,321

(22) Filed: Feb. 27, 2025

Related U.S. Application Data
(63) Continuation-in-part of application No. 17/496,118, filed on Oct. 7, 2021, now abandoned.
(60) Provisional application No. 63/088,590, filed on Oct. 7, 2020.

(51) Int. Cl. A63B 24/00; A63B 71/06

(52) U.S. Cl. CPC A63B 24/0075; A63B 24/0062; A63B 24/0084; A63B 24/0087; A63B 71/0622; A63B 71/0686; A63B 2024/0068; A63B 2024/0096; A63B 2071/063; A63B 2071/0691; A63B 2220/62; A63B 2225/20; A63B 2225/50

(57) ABSTRACT

A system and method for creating customized exercise routes through a mobile device interface enables users to build routes based on specific criteria. The system includes an exercise plan creating module for generating routes randomly, specifying start/end points, or manual drawing. Users can customize routes by selecting parameters including distance, topography, and terrain type, while incorporating landmarks and safety locations. The system provides route previews through multiple visualization formats with color-coded difficulty indicators. The system implements comprehensive data collection and analysis capabilities, with an artificial intelligence module providing personalized recommendations. Features include team management functionality, audio pacing capabilities that adjust to user performance, and a customizable avatar system for tracking fitness metrics. The system enables real-time route modifications while maintaining data accessibility even with limited connectivity, and includes emergency safety monitoring and data sharing capabilities between users.

### CROSS-REFERENCE TO RELATED APPLICATIONS

This application is a continuation-in-part of U.S. patent application Ser. No. 17/496,118, filed on Oct. 7, 2021, which claims the benefit of U.S. Provisional Application No. 63/088,590, filed on Oct. 7, 2020. The entire disclosures of the above applications are incorporated herein by reference.

### FIELD

The present technology relates to methods, systems, and storage media for creating a customized exercise plan.

### INTRODUCTION

This section provides background information related to the present disclosure which is not necessarily prior art.

People travel by a wide variety of means. Examples of such means include running, walking, hiking, biking, driving, and the like. Sometimes people travel for necessity, and sometimes for physical fitness, and other times for recreation. Indeed, people are known to travel for a variety of additional reasons.

There are numerous paths for people to travel. Such paths include roads, sidewalks, hiking trails, bike trails, and so on. Many of such paths are captured in maps. Maps showing the streets/highways located in a given area are common. Maps showing paths like bike trails and hiking trails also exist in a variety of forms. Many people are familiar with paper maps, which have been available for many years. Electronic maps have also emerged as a viable source for viewing several types of paths in recent decades.

People who travel often consult such maps. Sometimes, people consult maps before traveling in order to plan their travel routes. Other times, people consult maps after traveling in order to see where they have been and how far they have traveled. People may gain valuable information about their travel by consulting maps.

In addition to maps, people often consult other sources of information while making travel plans. For example, people may seek information related to weather, road construction, land-marks, and various other items of information. To gather this other information, people may be required to consult several sources.

Known types of maps, in both their paper and electronic forms, can be difficult to use for people who want to use customized routes for their travel, exercise or recreation. Such maps are often predetermined according to the platform being used, and may only suggest preset routes based on a limited number of factors not necessarily tailored to the needs or desires of the user.

There is a continuing need for a route building system and method that is easily accessible and provides desired information regarding the use of the generated routes. Desirably, the route building system and method permits for a superior customization of generated routes compared to known systems.

### SUMMARY

In concordance with the instant disclosure, a route building system and method that is easily accessible and provides desired information regarding the use of the generated routes, and which permits for a superior customization of generated routes compared to known systems, has been surprisingly discovered.

In one embodiment, the present disclosure relates to a method for creating a customized route to travel. The method may include providing a system for creating a customized route to travel, the system including a mobile device having a human interface for inputting user-determined criteria, a memory on which processor executable instructions may be stored, a processor in communication with the human interface and the memory, and a mobile display for displaying the customized route to travel compiled by the system server. The system server may be configured for analyzing the user-determined criteria and for generating the customized route to travel. A system server may be in communication with the mobile device through a wide area network. The method may include permitting the user, by human interface of the mobile device, to select the user-determined criteria including a duration of the exercise, a pattern of the exercise, a level of difficulty of the exercise, an inclusion of obstacles in the exercise, a general location of the exercise, and a presence of visual stimuli during exercise. The method may include transmitting the user-determined criteria from the mobile device to the system server. The method may include creating, by the system server, the customized route to travel based on user-determined criteria. The method may include transmitting the customized route to travel from the system server to the mobile device through the wide area network. The method may include generating a display of the customized route to travel on the mobile display of the mobile device, the display of the customized route to travel including a visual map of a route associated with the customized route to travel. The method may include providing, by the mobile device, real-time information to the user regarding the exercise plan including guidance and user feedback.

In another embodiment, the present disclosure relates to a system for creating a customized route to travel. The system may include one or more hardware processors configured by machine-readable instructions to create a customized route to travel. The machine-readable instructions may be configured to provide a system for creating a customized route to travel. The system may include a mobile device having a human interface for inputting user-determined criteria, a memory on which processor executable instructions be stored, a processor in communication with the human interface and the memory, and a mobile display for displaying the customized route to travel compiled by the system server. A system server may be configured for analyzing the user-determined criteria and for generating the customized route to travel, the system server in communication with the mobile device through a wide area network. The machine-readable instructions may be configured to permit the user, by human interface of the mobile device, to select the user-determined criteria including a duration of the exercise, a pattern of the exercise, a level of difficulty of the exercise, an inclusion of obstacles in the exercise, a general location of the exercise, and a presence of visual stimuli during exercise. The machine-readable instructions may be configured to transmit the user-determined criteria from the mobile device to the system server. The machine-readable instructions may be configured to create, by the system server, the customized route to travel based on user-determined criteria. The machine-readable instructions may be configured to transmit the customized route to travel from the system server to the mobile device through the wide area network. The machine-readable instructions may be configured to generate a display of the customized route to travel on the mobile display of the mobile device, the display of the customized route to travel including a visual map of a route associated with the customized route to travel. The machine-readable instructions may be configured to provide, by the mobile device, real-time information to the user regarding the exercise plan including guidance and user feedback.

In yet another embodiment, the present disclosure relates to a computer-readable storage medium for creating a customized route to travel. In some embodiments, the computer-readable storage medium may include instructions being executable by one or more processors for providing a system for creating a customized route to travel. The system may include a mobile device having a human interface for inputting user-determined criteria, a memory on which processor executable instructions may be stored, a processor in communication with the human interface and the memory, and a mobile display for displaying the customized route to travel compiled by the system server. A system server may be configured for analyzing the user-determined criteria and for generating the customized route to travel. The system server may be in communication with the mobile device through a wide area network. In some embodiments, the computer-readable storage medium may include instructions being executable by one or more processors to permit the user, by human interface of the mobile device, to select the user-determined criteria including a duration of the exercise, a pattern of the exercise, a level of difficulty of the exercise, an inclusion of obstacles in the exercise, a general location of the exercise, and a presence of visual stimuli during exercise. In some embodiments, the computer-readable storage medium may include instructions being executable by one or more processors to transmit the user-determined criteria from the mobile device to the system server. In some embodiments, the computer-readable storage medium may include instructions being executable by one or more processors to create, by the system server, the customized route to travel based on user-determined criteria.

In some embodiments, the computer-readable storage medium may include instructions being executable by one or more processors to transmit the customized route to travel from the system server to the mobile device through the wide area network. In some embodiments, the computer-readable storage medium may include instructions being executable by one or more processors to generate a display of the customized route to travel on the mobile display of the mobile device, the display of the customized route to travel including a visual map of a route associated with the customized route to travel.

In further embodiments, the computer-readable storage medium may include instructions being executable by one or more processors to provide, by the mobile device, real-time information to the user regarding the exercise plan including guidance and user feedback.

In exemplary embodiments, computerized route building systems for creating a customized route to travel may be constructed in accordance with the present technology may include a computer system and a computer-readable application. The computer system may have at least one processor which may further include at least one set of memory. The at least one set of memory may include tangible and non-transitory processor-readable media. The at least one processor may be configured to execute computer-readable instructions on its memory, including instructions for operation of the computerized route building system in accordance with the methods described herein.

The computerized route building system for creating a customized route to travel according to exemplary embodiments permits for the execution of a method according to the present disclosure. The method includes the steps of accessing the computerized route to travel system, which may further include downloading the computer application which retains the system, logging into the system via the internet, or retrieving a copy of the system stored on at least one article of hardware. Next, the method may also include a step of selecting at least one of a route from a pre-determined list of routes and a user designed route. Where the user designs their own route, a plurality of options may be selected such as route shape, topography, terrain, surroundings, and landmarks, as non-limiting examples. Then, the method may include a step of selecting a warm-up or cool-down. Afterwards, the method may include a step of reviewing a map of at least one of the designed and selected route. The method may also include a step of entering a goal which may be determined by at least one of an overall time to complete the route and a pace. Next, the method may include a step of selecting audio. The step of selecting audio may include at least one of a downloaded playlist, at least one of a pre-determined playlist and a customizable playlist that matches a desired beat of the audio file with at least one of an actual pace of a user and a goal pace of a user, and streaming an audio file such as an audiobook and podcasts, as a non-limiting examples.

In certain examples, the computerized route to travel building system may communicate between a plurality of devices. In further examples, the computerized route to travel building system may be synchronized between multiple devices of a single user, such as a smart phone and a smart watch, as non-limiting examples. In other examples, the computerized route to travel building system may communicate between user profiles of a plurality of users. The plurality of users may compare their pace to other users that utilize the computerized route to travel building system.

Once the application for the computerized route to travel building system is downloaded to a device of a user, the route may still be accessible, even where the device loses service or connectivity to satellites. Advantageously, the computerized route to travel building system may be used without the limitations of requiring connectivity to satellites or the usage of a user's data plan with their mobile device provider, as non-limiting examples. Desirably, the computerized route to travel building system may continuously provide the user with saved details about route, the terrain, and the weather.

In other embodiments, the computerized route to travel building system may include an artificial intelligence feature configured to provide recommendations based on the performance of the user. For example, if the user dramatically slowed their pace during a section of the route with more hills, the computerized route to travel building system may recommend more hill training workouts. In another example, if the user takes long breaks between training sessions due to soreness, the computerized route to travel building system may recommend at least one of an ice bath and particular stretches. In a more specific example, the artificial intelligence feature may monitor at least one of an external temperature and a temperature of a user delivered from a device of the user. The artificial intelligence feature may further deliver recommendations to the user to slow down where the least one of the external temperature and the temperature of a user is undesirable for continuing the route at a current pace. In another specific example, the artificial intelligence feature may track the total distance a user has used certain articles of equipment, such as at least one of the footwear and bicycle tires of the user, as non-limiting examples. The artificial intelligence feature may further recommend an appropriate opportunity to replace the article of equipment, such as based on the estimated insufficient tread on at least one of the shoes and the tires of the user. One skilled in the art may select other suitable methods of providing recommendations to users based on their selections, history, and performance, within the scope of the present disclosure.

In some embodiments, the artificial intelligence feature may monitor the location of the user from a Global Positioning System (GPS) feature on the device of the user. In a specific example, the location of the device of the user may be shared with people selected by the user, such as family or close friends, as non-limiting examples. In a more specific example, the artificial intelligence feature may monitor where the user has stopped for longer than a first predetermined period of time and longer than a second predetermined period of time during the route. Where the user has stopped for longer than the first predetermined period of time, an alert to call emergency services is delivered to the device of the user. Where the user has stopped for longer than the second predetermined period of time without dismissing the alert to call emergency services, the computerized route to travel building system may automatically call nearby emergency services and share the location of the device of the user with the emergency services. Advantageously, the location monitoring feature may provide a user with more confidence and peace of mind.

In particular examples, the artificial intelligence feature may be configured to provide real time recommendations to a user during the route. In a more particular example, the real time recommendations may be delivered through at least one of notifications on the device of the user and audio instructions through the device of the user. In an even more particular example, the real time recommendations may include at least one of motivational expressions, notifications such as changes in weather, pollen/air quality levels, traffic, estimated water loss, and personal coaching based on at least one of a goal time of the user, the performance of another selected user, and the computer generated “ghost partner” for personal competition based on the specific goals of the user. A skilled artisan may select other suitable methods for delivering real time recommendations to a user, within the scope of the present disclosure.

In other examples, the computerized route to travel building system may be adaptable during the route. In a more specific example, the route data may be adjusted in real time. For instance, the warm-up may be reduced, and the main route may be extended, as a non-limiting example. Additionally, the route may be abandoned and the quickest route to a pre-determined location may be available, as a non-limiting example. In specific examples, the computerized route building system may display and optionally provide directions to common points of interest such as nearest bathrooms, rest areas, and police stations, as non-limiting examples. Advantageously, the adaptable route feature may provide a user with more options to customize their route in real time. Desirably, the adaptable route feature may provide a user with more confidence to complete their route where they may need to shorten their route or find the quickest route to a desired location in real time.

In additional embodiments, users of the computerized route to travel building system may also share their routes and their performances to a community feature within the computerized route to travel building system. The community feature may be configured to allow users to share routes, challenges, workouts, articles/stories, digital images, tips, and recipes, as non-limiting examples. Advantageously, the community feature may increase the motivation and consistency of a user to continue utilizing training programs and more routes.

In particular examples, the device of the user may include at least one of a smartphone, tablet, smart watch, laptop, and other devices that may connect to the internet. The computerized route to travel building system may be accessed on the device of the user through at least one of a WIFI connection, a cellular satellite internet connection, and a hardline internet connection. The user may utilize the computerized route to travel building system through a plurality of menu screens displayed on a screen of the device of the user. A user of the computerized route to travel building system may connect with another user of the computerized route to travel building system where the devices of the users are connected to at least one of a WIFI connection, a cellular satellite internet connection, and a hardline internet connection. Each user of the computerized route to travel building system may simultaneously connect with a plurality of other users of the computerized route to travel building system.

Advantageously, the system is easily accessible and provides desired information regarding the use of the generated routes to a user in one location.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a schematic illustration of a system configured for creating a customized route to travel according to one embodiment of the present disclosure;

FIG. 2 is a flowchart showing a method for creating a customized route to travel according to one embodiment of the present disclosure;

FIG. 3 is another flowchart showing a method for creating a customized route to travel according to one embodiment of the present disclosure;

FIG. 4 is a detailed flowchart showing the step of permitting the user to select the user-determined criteria from FIG. 2;

FIG. 5 is screenshot showing a customized route to travel according to one embodiment of the present disclosure;

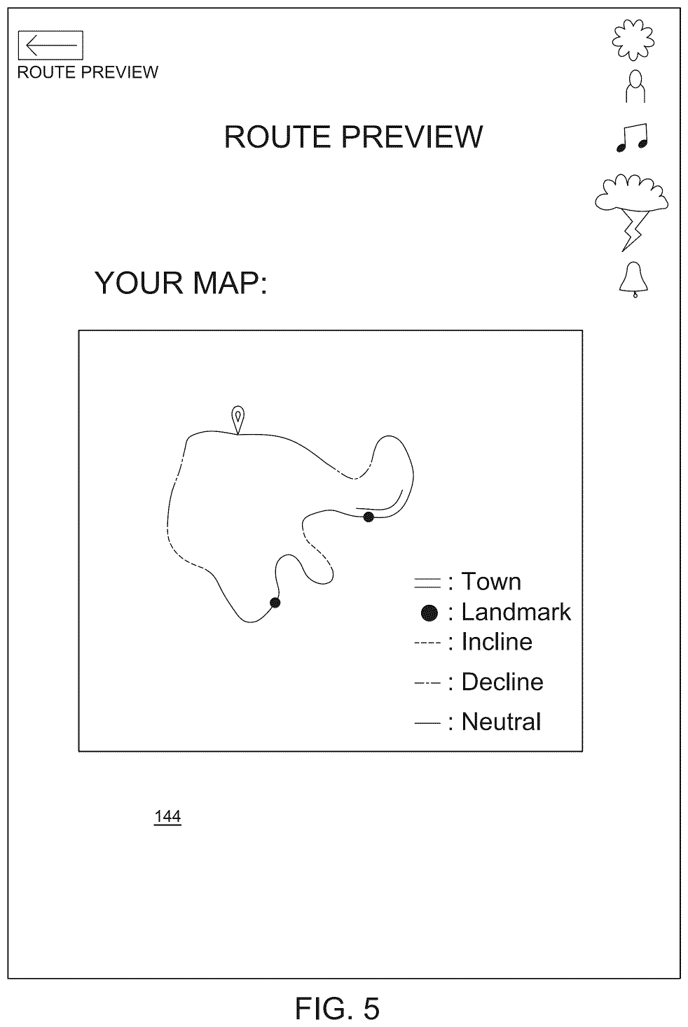

FIG. 6 is another screenshot showing a customized route to travel according to one embodiment of the present disclosure;

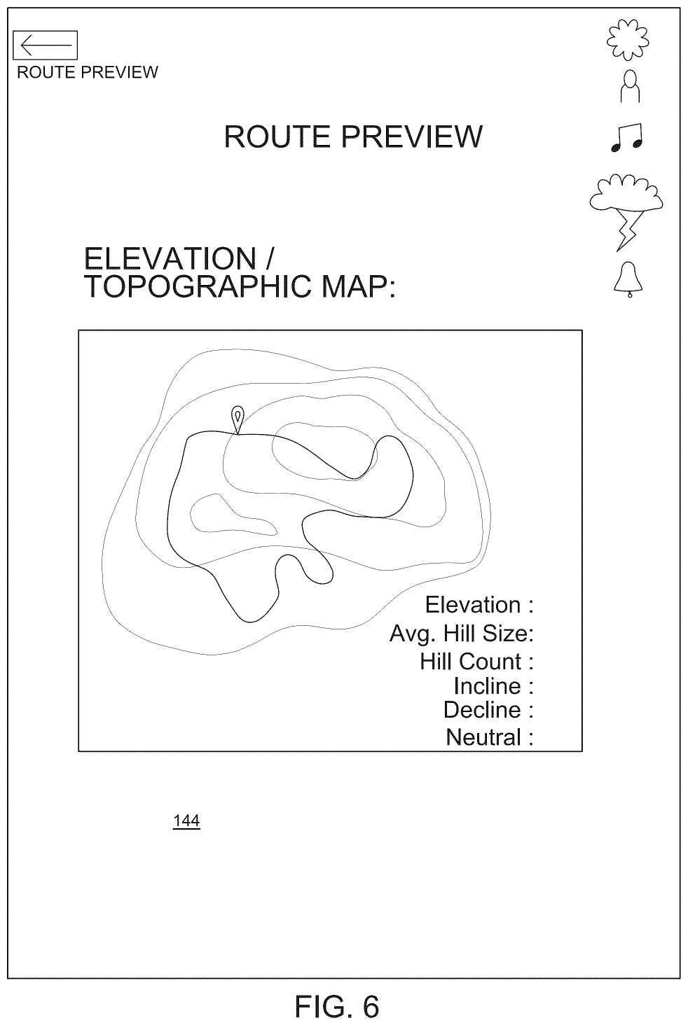

FIG. 7 is another screenshot showing a customized route to travel according to one embodiment of the present disclosure.

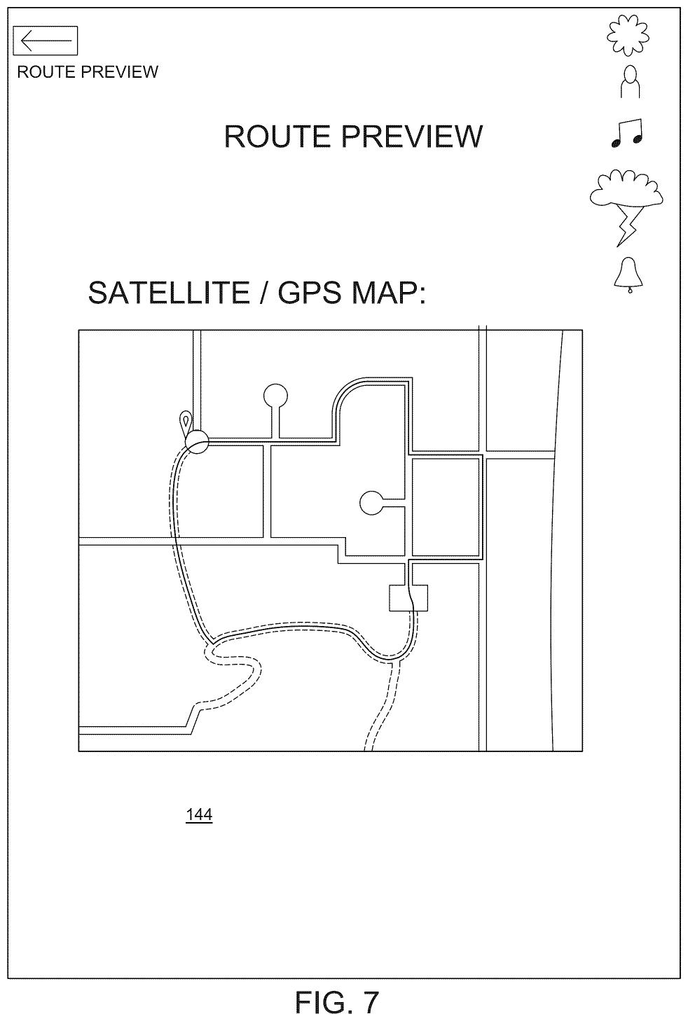

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps may be different in various embodiments, including where certain steps may be simultaneously performed, unless expressly stated otherwise.

“A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” may encompass both an orientation of above and below. The device may be otherwise oriented (rotated 90 degrees or at other orientations) and the spatially relative descriptors used herein interpreted accordingly.

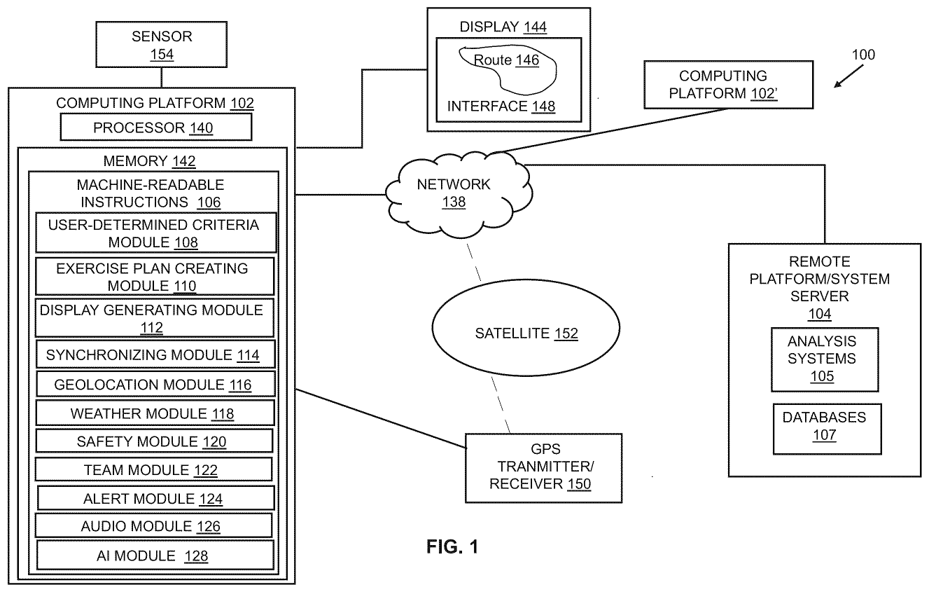

FIG. 1 illustrates a system 100 configured for creating a customized route to travel, for example, as part of a customized exercise plan, in accordance with one or more embodiments.

The system 100 may include one or more computing platforms 102. The computing platforms 102 may be provided in the form of one or more mobile devices such as smartphones, tablet computers, or the like. The one or more computing platforms 102 may be communicably coupled with one or more remote platforms/system server 104. In some cases, users may access the system 100 via remote platforms/system server(s) 104. The one or more computing platforms 102 has a processor 140 and a memory 142, as described in further detail hereinbelow.

The one or more computing platforms 102 may be configured by machine-readable instructions 106 that are stored on the memory 142 of the one or more computing platforms 102. Machine-readable instructions 106 may include modules. The modules may be implemented as one or more of functional logic, hardware logic, electronic circuitry, software modules, and the like. As shown in FIG. 1, the modules may include one or more of user- determined criteria module 108, exercise plan creating module 110, display generating module 112, a synchronizing module 114, a geolocation module 116, a weather module 118, safety module 120, a team module 122, an alert module 124, an audio module 126, artificial intelligence module 128, and/or other modules. It should be appreciated that one skilled in the art may select other suitable modules for use with the one or more computing platforms 102 within the scope of the present disclosure.

The computing platform 102 may be configured to include a security module for limiting access to the computing platform 102. The security module may include features to limit unauthorized access to the computing platform 102. The features of the security module may include any features known to those of skill in the art for limiting access to a computing platform 102. Non-limiting examples include, but are not limited to, face recognition, fingerprint sensors, security code, two-step authentication, biometric security features, and other security features known to those of skill in the art.

The computing platform 102, after access has been granted, may be configured to implement one of several modules for capturing information for use in generating the customized route to travel. The modules may all be included or select modules may be implemented, as desired.

The user-determined criteria module 108 may include a variety of exercise related criteria, as a non-limiting example. These criteria may include, but are not limited to, type of exercise, amount of customization, duration of exercise, a pattern of the exercise, a level of difficulty of the exercise, an inclusion of obstacles in the exercise, a general location of the exercise, and a presence of visual stimuli during exercise. The user-determined criteria module 108 may be configured to transmit the user-determined criteria from the mobile device to the system server 104.

More specifically, the user-determined criteria module 108 allows the user to select the type of exercise to be completed by the user. Non-limiting examples of such types of exercise include, but are not limited to, walking, running, cycling, swimming, hiking, and combinations of these exercises.

The user-determined criteria module 108 may further be configured to allow the user to select a duration of the exercise. The duration may be chosen from a length of time, a distance, calories burned, or other similar defining factor.

The user-determined criteria module 108 may be configured to allow the user to select the pattern of the customized route to travel. Non-limiting examples of such patterns include, but are not limited to, a loop, box, out-and-back, and a random selection.

The user-determined criteria module 108 may be configured to allow the user to select the topography for the customized route to travel. Non-limiting examples of such topography include, but are not limited to, small hills, medium hills, large hills, increasing incline, flat surface, and combinations thereof.

The user-determined criteria module 108 may be configured to allow the user to select the surface on which they may be performing the customized route to travel. Non-limiting examples of such surface styles include, but are not limited to, gravel, paved roads, sidewalks, grass, track, and combinations thereof.

The user-determined criteria module 108 may be configured to allow the user to select the environment in which the user may perform the customized route to travel. Non-limited examples include, but are not limited to, in a forest, in a park, in a city, in a town, off-road, and a random selection.

The user-determined criteria module 108 may be configured to allow the user to select the visual stimuli that the user may see while performing the customized route to travel. Non-limited examples include, but are not limited to, landmarks, bodies of water, famous sites, and other similar visual stimuli.

The user-determined criteria module 108 may be configured to allow the user to add a warmup, cooldown, or both a warmup and cooldown to the customized route to travel.

The exercise plan creating module 110 may be configured to create, by the system server 104, the customized route to travel based on user-determined criteria. The exercise plan creating module 110 may be configured to transmit the customized route to travel from the system server 104 to the computing platform 102 such as the mobile device through the wide area network or other similar transmissions.

The display generating module 112 may be configured to generate a display 144 of the customized route to travel on the mobile display of the computing platform 102 such as the mobile device. The display 144 of the customized route to travel may include a visual map 146 of a route associated with the customized route to travel. The display 144 may be 2-dimensional, a 3-dimensional or a rotatable 3-dimensional display of the customized route to travel that may be viewed on the mobile display of the computing platform 102, using systems known to those of skill in the art. The display generating module 112 may also provide elevational and topological detail of the customized route to travel on the mobile display of the computing platform 102. The display generating module 112 may also provide detailed information regarding the customized route to travel. This detailed information may include, but is not limited to, percent (%) incline, percent (%) of a specific surface (e.g., grass, paved road, gravel), weather conditions, and other similar detailed information.

In other examples, the display generating module 112 may also enable the user, by the human interface 148 of the computing platform 102, to alter the exercise plan during use of the customized route to travel and may provide real-time information to the user regarding the exercise plan including guidance and user feedback. In a more specific example, the customized exercise plan data may be adjusted in real time. For instance, additional feature may be added, such as a warm-up and the duration may be extended, as a non-limiting example. Additionally, the customized route to travel may be cancelled and the quickest route to a pre-determined location (such as the start of the route) may be available, as a non-limiting example. Advantageously, the adaptable route feature may provide a user with more options to customize their customized route to travel in real time. Desirably, the adaptable route feature may provide a user with more confidence to complete their route where they may need to shorten their route or find the quickest route to a desired location in real time.

In some cases, the system 100 may further include a synchronizing module 114 configured to automatically synchronize all information related to the customized route to travel to the system server 104. The synchronizing module 114 may include synchronizing the customized route to travel created, performance data, times, weather conditions, music and any other material included as part of a customized route to travel. The route data that may be both stored and synchronized may include the following non-limiting examples: total distance of rout, average pace, pace per mile/kilometer, average MPH/KPH, MPH/KPH per each mile/kilometer, start time, end time, duration of route, time of each mile/kilometer, average heart rate, heart rate of each mile/kilometer, temperature during route, weather during route, total percent of incline/decline, percent of incline/decline of each mile/kilometer, average route elevation, elevation of each mile/kilometer, set goal, whether the goal was met/not met/surpassed, route conditions, overall terrain, terrain of each mile/kilometer, total calories burned, calories burned of each mile/kilometer, performance rating (poor, average, good), route level of difficulty, performance of each mile/kilometer (poor, struggled, excelled, increased, slowed down), overall performance rating. This information may be stored as raw data or in graphical representation. The graphical representations may include comparisons to prior activities.

In some cases, the system 100 may further include at least one geolocation module 116, such as a global positioning system (GPS), in communication with at least one of the computing platform 102 and the system server 104, and the step of transmitting the customized route to travel includes determining a geolocation of the computing platform 102 such as the mobile device within the customized route to travel. Thus, the computing platform 102, such as the mobile device, may include a transmitter/receiver 150 enabling communication with a geolocation system, such as a GPS, that may communicate via a satellite 152.

The system 100 may further include a weather module 118. The weather module 118 may provide the forecast for the location selected by the user-determined criteria. Additionally, the weather module 118 may provide clothing recommendations, possible routes conditions (c.g., icy, muddy, etc.),traffic updates, and pollen and air quality reports.

The system 100 may further include at least one sensor 154 in communication with the computing platform 102 such as the mobile device. The sensor 154 may be configured to assess and communicate to the system server 104 one or more user attributes selected from the group consisting of body temperature, hydration, and pulse. Non-limiting examples of such attributes include, but are not limited to, body temperature, hydration, blood pressure, and pulse. The user attributes information may be relayed to the system and the attribute may be quantified. This assessment may be compared against a predetermined acceptable range, such that if the assessed user attribute quantification falls outside of the predetermined acceptable range an alert may be provided to the user on the mobile display of the mobile device. Nonlimiting examples of the sensors include, but are not limited to, accelerometers, gyroscopes, electromagnetic sensors, biological sensors, and optical tracking sensors. It should further be appreciated that a skilled artisan may employ different types and numbers of the sensors 154 as required by the procedure or situation within which the system 100 is being used.

The sensors may also be used to monitor user movement. The sensors may be configured to detect prolonged stops or breaks during a customized route to travel. Detection of a prolonged stop triggers an alert to the user on the mobile display of the computing platform 102 such as the mobile device. The user must then provide an interaction with the computing platform 102 within a predetermined amount of time, such that absent such an interaction an emergency alert may be sent by the computing platform 102 to emergency personnel that are in close proximity to the user based on geolocation information.

In a specific example, the computing platform 102 location may be shared via the network with one or more other computing platforms 102 on mobile devices of third party users also running the system 100, such as the family or friends of the user', such as family or close friends, as non-limiting examples. In a more specific example, the system 100 may monitor where the user has stopped for longer than a first predetermined period of time during the customized route to travel, such as if the user stops for over three (3) minutes. Where the user has stopped for longer than the first predetermined period of time, an alert to call emergency services is delivered to the computing platform 102 of the user. Where the user takes longer than the second predetermined period of time, such as 20 seconds, without dismissing the alert to call emergency services, the system 100 may automatically call nearby emergency services and share the location of the user with the emergency services. Advantageously, the location monitoring feature may provide a user with more confidence and peace of mind.

The system 100 may further include the safety module 120 configured that may be initiated by the user during performance of the customized route to travel. In some cases, the safety module 120 may be configured to activate a tracking system for determining a user proximity to police, fire department, and bathrooms.

The system 100 may further include a team module 122 configured to allow groups of people to share exercise plan information, compare training routines, and compete with each other. The team module 122 may be configured to allow users to sign up for a group and share information using another computing platform 102′ on another mobile device. Non-limiting examples of such information include, but are not limited to, synchronized training/exercise information, music, compare training routines, create competitions, share recipes, share pictures, and create a scrapbook. Additionally, the data may include the following non-limiting examples, which can be compared between other team module users: total distance of route, average pace, pace per mile/kilometer, average MPH/KPH, MPH/KPH per cach mile/kilometer, start time, end time, duration of route, time of each mile/kilometer, average heart rate, heart rate of each mile/kilometer, temperature during route, weather during route, total percent of incline/decline, percent of incline/decline of each mile/kilometer, average route elevation, elevation of each mile/kilometer, set goal, whether the goal was met/not met/surpassed, route conditions, overall terrain, terrain of each mile/kilometer, total calories burned, calories burned of each mile/kilometer, performance rating (poor, average, good), route level of difficulty, performance of each mile/kilometer (poor, struggled, excelled, increased, slowed down), overall performance rating. This information may be stored as raw data or in graphical representation. The graphical representations may include comparisons to prior activities. The team module 122 may also be configured to provide information about nearby clubs or events related to the exercise.

In certain examples, the system 100 may communicate between a plurality of devices. In further examples, the system 100 may be synchronized, using the synchronizing module 114, between multiple devices of a single user, such as a smart phone and a smart watch, as non-limiting examples. In other examples, the system 100 may communicate between user profiles of a plurality of users. For example, a plurality of users may compare their pace to other users that utilize the system. Advantageously, the community feature may increase the motivation and consistency of a user to continue utilizing training programs and more routes.

The system 100 may further include an alert module 124 configured to assess the customized exercise training plan, assess the performance of the customized training plan, and provide alerts for potential training problems such as overtraining and tire or shoe tread wear. The alert module 124 may provide warnings to the user about such training problems and offer training suggestions, physical therapists, local stores with equipment, and other similar recommendations. One skilled in the art may select other suitable recommendations to users based on their selections, history, and performance, within the scope of the present disclosure.

In some cases, the system 100 may further include an audio module 126 configured to enable audio that may be integrated into the customized route to travel. Non-limiting examples of such audio include, but are not limited to include music, podcasts, books, and playlists. The audio module 126 may also include smart beat technology that works within the customized route to travel. The smart beat technology may match the pace needed for the user to achieve the goal selected in the user-determined criteria 108, such that if the user is falling off of the required pace the beat will speed up until the required pace is achieved. Alternatively, the smart beat technology may either match your pace or maintain a constant pace. Alternatively, the audio module 126 may include a voice option/audio coach that may provide motivation, instruction, and directions during performance of the customized route to travel. The coach option may include different voice options.

The system 100 may also include an artificial intelligence (AI) or machine learning module 128, using standard machine learning systems know to those of skill in the art, which is configured to receive data regarding performance of the customized route to travel, assess the performance, and provide feedback regarding the performance. For example, the AI module 128 may provide post route recommendations, such as shorten your stride at certain points, lengthen stride at certain points, or increase muscles to increase performance. Additionally, the AI module 128 may adjust the customized route to travel for future uses based on past performance. For example, if the user struggled with hills, the AI module 128 may offer route suggestions to improve performances in those locations where the user struggled, or the AI module 128 may create a training plan to increase the muscles needed for better performance in the locations where the user struggled.

In other embodiments, the AI module 128 may be configured to analyze data compiled from the exercise plan and may modifying future customized route to travels from the analysis of the user data collected during the customized route to travel, the modifications being selected from route recommendations, including a warmup, including a cool-down, and altering route difficulties, the step of analyzing data further includes recommending technical exercise improvements selected from the group consisting of altering customized route to travel routes, post route workout advice, and recovery techniques and the analyzing step further includes generating post workout graphs and charts. The data that may be analyzed may include the following non-limiting examples: total distance of rout, average pace, pace per mile/kilometer, average MPH/KPH, MPH/KPH per each mile/kilometer, start time, end time, duration of route, time of each mile/kilometer, average heart rate, heart rate of each mile/kilometer, temperature during route, weather during route, total percent of incline/decline, percent of incline/decline of each mile/kilometer, average route elevation, elevation of each mile/kilometer, set goal, whether the goal was met/not met/surpassed, route conditions, overall terrain, terrain of each mile/kilometer, total calories burned, calories burned of each mile/kilometer, performance rating (poor, average, good), route level of difficulty, performance of each mile/kilometer (poor, struggled, excelled, increased, slowed down), overall performance rating. This information may be stored as raw data or in graphical representation. The graphical representations may include comparisons to prior activities.

With respect to the warmup and cooldown, it should be appreciated that the user may add the warmup or the cooldown to the main route. If the user adds this to the main route, the user may set up a desired distance. When that desired distance is reached the data may be saved separately from the main route. If the user ends a warmup or cooldown early, or if the user goes longer, the user can go into the system 100 after the route is uploaded and correct the warmup or cooldown distances. The system 100 may then automatically readjust the data and statistics.

Alternatively, the user may select to keep the warmup or cooldown separate from the main route. In this case, the user can add specifications, but the warmup or cooldown will not be attached to the main route or start and finish in a location within the system away from the main route. The data collected will not be incorporated or affect the data collected from the main route. The data sets will remain separate within the system 100 in this case.

It should be understood that the post-workout graphs may be visual graphics on the mobile display to depict (c.g., using color coding) for the user the areas in need of additional strengthening. The graphics may include a variety of graph analytics. The graph analytics may include color coding to show areas of difficulty. The AI module 128 may also be configured to create a ghost run to provide the user with a preview of potential performance on a customized route to travel. For example, the user may input their fastest time into the module. This then may allow the user to compete against their fastest time on a specific route. The ghost run may be based on collected data on previous routes or just input in as a time (e.g., the time can be broken down specifically per mile or kilometer, i.e. 6 min. first mile, 5 min. second mile).

In other embodiments, the AI module 128 may be configured to provide recommendations based on the performance of the user. For example, if the user dramatically slowed their pace during a section of the route with more hills, the AI module 128 may recommend more hill training workouts. In another example, if the user takes long breaks between training sessions due to soreness, the AI module 128 may recommend at least one of an ice bath and particular stretches. In a more specific example, the AI module 128 may monitor, in connection with the sensors, at least one of an external temperature and a temperature of a user delivered from a device of the user. The AI module 128 may further deliver recommendations to the user to slow down where the least one of the external temperature and the temperature of a user is undesirable for continuing the customized route to travel at a current pace. In another specific example, the AI module 128 may track the total distance a user has used certain articles of equipment, such as at least one of the footwear and bicycle tires of the user, as non-limiting examples. Other non-limiting examples of recommendations include, but are not limited to, more routes with hills, strengthen specific muscles, longer strides, shorter strides, increase lung capacity and methods of doing so such as breathing exercises, altitude training, specific workouts, cat more calories, food and beverage recommendations, more water consumption, use of ice baths, stretching exercises, more incline training, less inclines, terrain recommendations, recommend goals, places where user struggled or excelled, recommendations for timing of breaks.

In particular examples, the AI module 128 may be configured to provide real time recommendations to a user during the customized route to travel. In a more particular example, the real time recommendations may be delivered through at least one of notifications on the mobile display of the mobile device of the user and audio instructions through the device of the user. In an even more particular example, the real time recommendations may include at least one of motivational expressions, notifications such as changes in weather, pollen/air quality levels, traffic, estimated water loss, and personal coaching based on at least one of a goal time of the user, the performance of another selected user, and the computer generated “ghost partner” for personal competition based on the specific goals of the user. A skilled artisan may select other suitable methods for delivering real time recommendations to a user, within the scope of the present disclosure.

Additionally, once the customized route to travel is downloaded to a device of a user, the customized route to travel may still be accessible, even where the device loses service or connectivity to satellites. Advantageously, the system 100 may be used without the limitations of requiring connectivity to satellites or the usage of a user's data plan with their mobile device provider, as non-limiting examples. Desirably, the system 100 may continuously provide the user with saved details about route, the terrain, and the weather.

In some cases, the one or more computing platforms 102, may be communicatively coupled to the remote platforms/system server(s) 104. In some cases, the communicative coupling may include communicative coupling through a networked environment 138. The networked environment 138 may be a radio access network, such as LTE or 5G, a local area network (LAN), a wide area network (WAN) such as the Internet, or wireless LAN (WLAN), for example. It will be appreciated that this is not intended to be limiting, and that the scope of this disclosure includes implementations in which one or more computing platforms 102 and remote platforms/system server(s) 104 may be operatively linked via some other communication coupling. The one or more one or more computing platforms 102 may be configured to communicate with the networked environment 138 via wireless or wired connections. In addition, in an embodiment, the one or more computing platforms 102 may be configured to communicate directly with each other via wireless or wired connections. Examples of one or more computing platforms 102 may include, but is not limited to, smartphones, wearable devices, tablets, laptop computers, desktop computers, Internet of Things (IOT) device, or other mobile or stationary devices. In an embodiment, system 100 may also include one or more hosts or servers, such as the one or more remote platforms/system servers 104 connected to the networked environment 138 through wireless or wired connections. According to one embodiment, remote platforms/system servers 104 may be implemented in or function as base stations (which may also be referred to as Node Bs or evolved Node Bs (eNBs)). In other embodiments, remote platforms/system servers 104 may include web servers, mail servers, application servers, etc. According to certain embodiments, remote platforms/system servers 104 may be standalone servers, networked servers, or an array of servers.

In one embodiment the computing platform 102 may be a mobile device. The mobile device may have a human interface for inputting user-determined criteria into the module user-determined module 108. The mobile device may include a mobile display for displaying the customized route to travel compiled by the system server 104. The display may be a liquid crystal display or other display known to those of skill in the art to be used in connection with the method and system of the present disclosure. The display may also include controllers for use by the user in inputting user-determined criteria into the system 100. Non-limiting examples of such controllers include, but are not limited to, a touch pad/screen, a switch, voice commands mechanisms, and other user interface mechanisms.

The one or more computing platforms 102 may include one or more processors 140 for processing information and executing instructions or operations. One or more processors 140 may be any type of general or specific purpose processor. In some cases, multiple processors 140 may be utilized according to other embodiments. In fact, the one or more processors 140 may include one or more of general-purpose computers, special purpose computers, microprocessors, digital signal processors (DSPs), field-programmable gate arrays (FPGAs), application-specific integrated circuits (ASICs), and processors based on a multi-core processor architecture, as examples. In some cases, the one or more processors 140 may be remote from the one or more computing platforms 102, such as disposed within a remote platforms/system server 104 like the one or more remote platforms/system servers 104 of FIG. 1.

The one or more processors 140 may perform functions associated with the operation of system 100 which may include, for example, precoding of antenna gain/phase parameters, encoding and decoding of individual bits forming a communication message, formatting of information, and overall control of the one or more computing platforms 102, including processes related to management of communication resources.

The one or more computing platforms 102 may further include or be coupled to a memory 142 (internal or external), which may be coupled to one or more processors 140, for storing information and instructions that may be executed by one or more processors 140. Memory 142 may be one or more memories and of any type suitable to the local application environment and may be implemented using any suitable volatile or nonvolatile data storage technology such as a semiconductor-based memory device, a magnetic memory device and system, an optical memory device and system, fixed memory, and removable memory. For example, memory 142 may consist of any combination of random-access memory (RAM), read-only memory (ROM), static storage such as a magnetic or optical disk, hard disk drive (HDD), or any other type of non-transitory machine or computer readable media. The instructions stored in memory 142 may include program instructions or computer program code that, when executed by one or more processors 140, enable the one or more computing platforms 102 to perform tasks as described herein.

In some embodiments, one or more computing platforms 102 may also include or be coupled to one or more antennas for transmitting and receiving signals and/or data to and from one or more computing platforms 102. The one or more antennas may be configured to communicate via, for example, a plurality of radio interfaces that may be coupled to the one or more antennas. The radio interfaces may correspond to a plurality of radio access technologies including one or more of LTE, 5 G, WLAN, Bluetooth, near field communication (NFC), radio frequency identifier (RFID), ultrawideband (UWB), and the like. The radio interface may include components, such as filters, converters (for example, digital-to-analog converters and the like), mappers, a Fast Fourier Transform (FFT) module, and the like, to generate symbols for a transmission via one or more downlinks and to receive symbols (for example, via an uplink).

The remote platforms/system server 104 may include analysis systems 105 to analyze the user-determined criteria, and other inputs by the user and in communication with the computing platform 102. The remote platforms/system server 104 may include databases of information 107. The information may include any of the information needed to be accessed by the system 100. The databases 107 may be used, in conjunction with the user-determined criteria, to generate or create the customized route to travel. The analysis system 105 contained within the remote platforms/system server 104 may jointly analyze the information stored within the databases 107 and the user-determined criteria to transform the data and user-determined criteria into an executable customized route to travel. This analysis creates a customized route to travel that may be transmitted to the user on the mobile display.

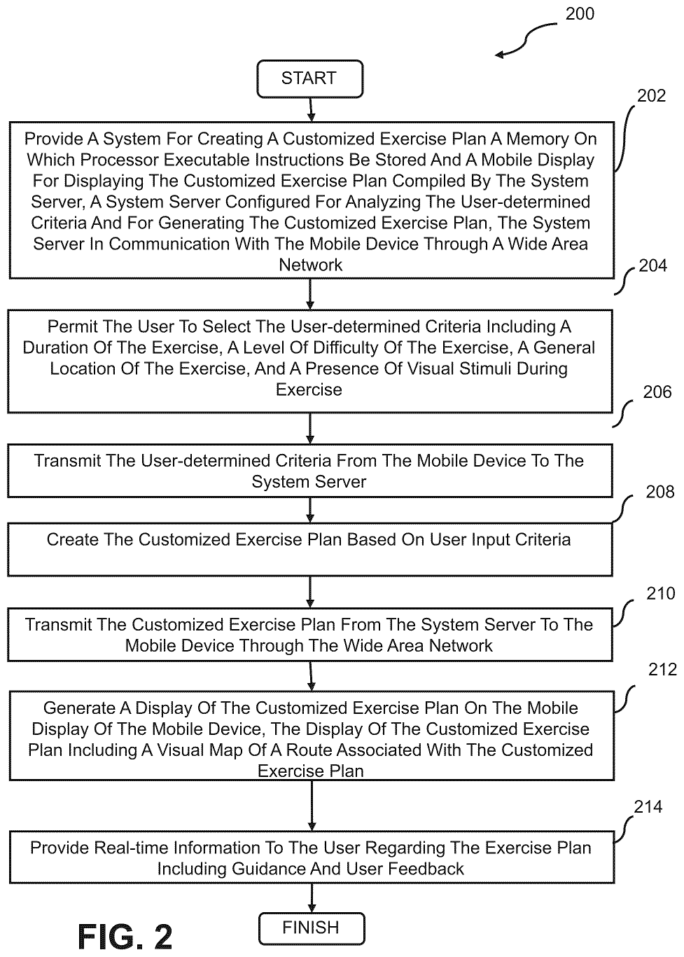

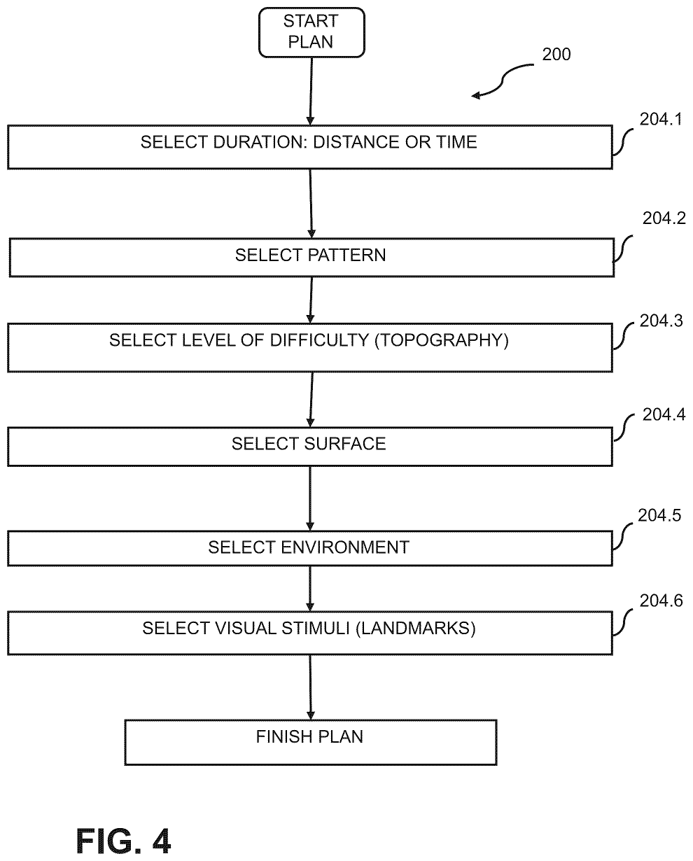

FIGS. 2 and 4 illustrates an example flow diagram of a method 200, according to one embodiment. The method 200 may include providing a system for creating a customized route to travel, the system including a mobile device having a human interface for inputting user-determined criteria, a memory on which processor executable instructions being stored, a processor in communication with the human interface and the memory, and a mobile display for displaying the customized route to travel compiled by the system server, a system server configured for analyzing the user-determined criteria and for generating the customized route to travel, the system server in communication with the mobile device through a wide area network at block 202. The method 200 may include permitting the user, by human interface of the mobile device, to select the user-determined criteria including a duration of the exercise at block 204.1, a pattern of the exercise at block 204.2, a level of difficulty (i.e. the topography) of the exercise at block 204.3, an inclusion of obstacles/selection of surface of the exercise at block 204.4, a general location or environment of the exercise at block 204.5, and a presence of visual stimuli (i.c. landmarks) during exercise at block 204.6. The method 200 may include transmitting the user-determined criteria from the mobile device to the system server at block 206. The method 200 may include creating, by the system server, the customized route to travel based on user-determined criteria at block 208. The method 200 may include transmitting the customized route to travel from the system server to the mobile device through the wide area network at block 210. The method 200 may include generating a display of the customized route to travel on the mobile display of the mobile device, the display of the customized route to travel including a visual map of a route associated with the customized route to travel at block 212. The method 200 may include providing, by the mobile device, real-time information to the user regarding the exercise plan including guidance and user feedback at block 214.

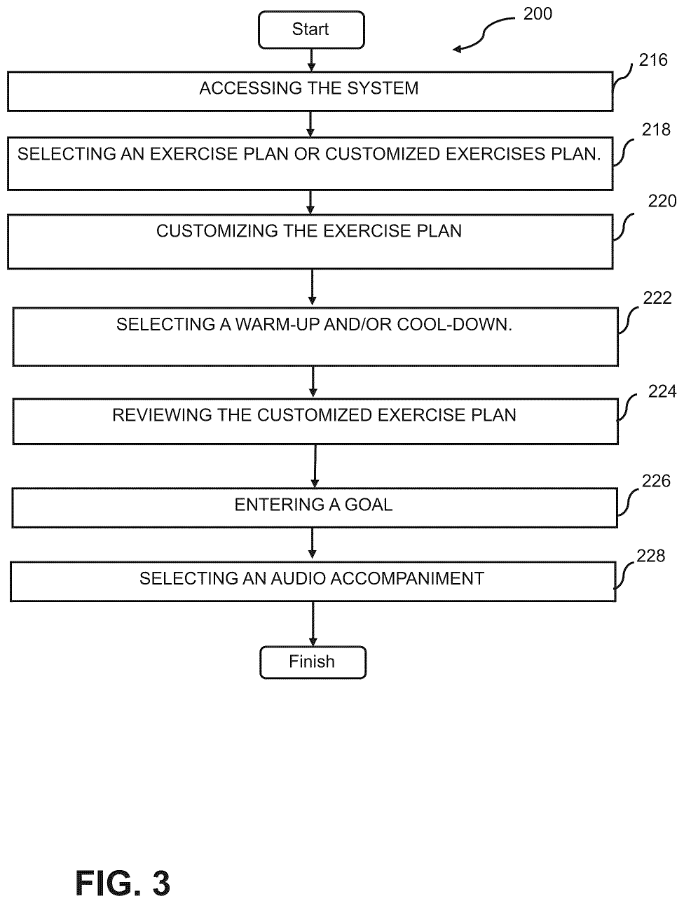

In particular embodiments, as shown in FIG. 3 the system may be configured to perform various operations that may be defined as method steps related to building one or more customized route to travels. Such methods may include the steps of accessing the system at block 216, which may further include downloading a computer application which retains the system, logging into the system via the internet, or retrieving a copy of the system stored on at least one article of hardware. Next, the method may also include a step of selecting at least one of an exercise plan from a pre-determined list of exercise plans and a customized route to travel at block 218. Where the user designs their own customized route to travel at block 220, a plurality of options may be selected such as route shape, topography, terrain, surroundings, and landmarks, as non-limiting examples. Then, the method may include a step of selecting a warm-up or cool-down at block 222. Afterwards, the method may include a step of reviewing a map of at least one of the designed and selected exercise plan at block 224. The method may also include a step of entering a goal which may be determined by at least one of an overall time to complete the route and a pace at block 226. Next, the method may include a step of selecting audio at block 228. The step of selecting audio may include at least one of a downloaded playlist, a pre-determined playlist that matches the beat with a pace of a user, and streaming an audio file such as an audiobook, as a non-limiting examples.

In a certain example, the method may include communicating between a plurality of devices. In specific examples, the system may be synchronized between multiple devices of a single user, such as a smart phone and a smart watch, as non-limiting examples. In other examples, the method may include communicating between user profiles of a plurality of users, wherein the users may compare their pace to other users that utilize the system for creating a customized route to travel.

Another embodiment of the system 100 can include the components and modules shown in FIG. 1-including at least the computing platform 102, remote platform/system server 104, analysis systems 105, and databases 107. The system 100 includes additional modules for implementing enhanced functionality.

In some embodiments, the exercise plan creating module 110 of system 100, in coordination with the system server 104 and the analysis systems 105 and databases 107, includes enhanced route building functionality that allows users to build customized routes based on specific criteria. Users can select a starting point, which may be their current location, a searched location, or a location selected on a map. The route building process allows a user to specify criteria including total route distance, route shape (such as loop, box, out and back, and combinations thereof), topography (such as flat, rolling hills, large hills, increasing incline, and combinations thereof), and terrain (such as paths, roads, grass, or combinations thereof). After a route is generated, users can modify the route by adding parts, removing parts, or redrawing/redesigning sections to better fit their preferences.

The exercise plan creating module 110′ of system 100′, in coordination with the artificial intelligence module 128, system server 104, and analysis systems 105 and databases 107, can include enhanced route building and recommendation functionality that can allow users to build customized routes based on specific criteria and receive AI-powered suggestions. Users can select a starting point, which can be their current location, a searched location, or a location selected on a map. The route building process can allow users to specify criteria including total route distance, route shape (such as loop, box, out-and-back configurations), topography (such as flat, rolling hills, large hills, increasing incline), and terrain (such as paths, roads, grass, or combinations thereof). The AI module 128 can analyze user performance data and fitness levels to generate personalized route recommendations and modifications. After a route is generated, users can modify the route by adding parts, removing parts, or redrawing sections to better fit their preferences. The Al module can provide post-route recommendations based on performance analysis, such as suggestions for improving technique on hills or adjusting stride length at certain points. The system can enable comprehensive data comparison and performance analysis through numerical or graphical representations, with the AI module generating recommendations for improvement based on analyzed data, statistics, and performance metrics while considering factors including goals, conditions, route difficulty, past routes, and current fitness level.

The exercise plan creating module 110 includes functionality for handling situations where user-requested route criteria are unavailable within a desired area. In such cases, the module provides users with multiple options: (1) locate the nearest route that matches the desired criteria and receive directions to that location, (2) view the additional distance required to reach a matching route and choose whether to incorporate that additional distance, or (3) generate an alternative route within the original area using only criteria that are available in that location. When showing the nearest matching route option, the module can display how far away the route is and can provide directions to reach the route. If the user chooses to incorporate the additional distance to reach a matching route, the user can adjust the route accordingly, including modifying warmups and cooldowns as needed.

The exercise plan creating module 110 can provide area blocking functionality that allows users to designate certain areas and locations for specific handling during route generation. Users can block off areas either to ensure routes stay specifically within the designated areas or to ensure routes completely avoid them. The blocked off areas can apply universally across all route generation methods, with the exercise plan creating module 110 respecting the area designations according to user preferences.

The exercise plan creating module 110 can provide comprehensive route preview and visualization capabilities. Users can preview routes through multiple formats including 2D maps, 3D maps, elevation/topographic maps, and satellite/GPS maps. The preview can include detailed route features and descriptions including route conditions, pictures, area reviews, traffic patterns, and terrain/topography breakdowns. The system breaks down route terrain by specific percentages for each type of surface (for example: 50% dirt, 25% grass, 20% pavement, 5% other) and provides detailed topography analysis showing the distribution of flat sections, hills, and inclines throughout the route. A map legend can identify key features including restrooms, paths, roads, police/fire stations, start/end points, inclines/declines, busy areas, and hazards. The system 100 can show the percentage match between generated routes and requested criteria, with difficult sections (like steep inclines), easy sections (like steady downhills), and neutral sections (like rolling hills) being color-coded both in preview and during active routes. When user-requested criteria are unavailable within a desired area, the exercise plan creating module 110 can provide multiple options. The exercise plan creating module 110 can locate and provide directions to the nearest matching route, calculate additional distance required to reach a matching route, or generate an alternative route using only locally available criteria. Users can edit selected routes by adding parts, removing sections, or redrawing portions. Routes can be started immediately, saved for later use, or shared with other users including teams, coaches, clubs, and the community.

The exercise plan creating module 110 can provide multiple distinct methods for route creation and generation. Users can generate routes through random generation (where the module applies user-specified criteria to create a randomized route), or through custom generation where users can specify start and end points for route creation. Users can also locate nearby existing routes, manually draw custom routes, or perform free runs where no predetermined route is required. For randomly generated routes, the exercise plan creating module 110′ applies any user-specified criteria and area restrictions while creating the route. When users choose custom generation by specifying start and end points, the module generates route options connecting those locations. The nearby route feature allows users to discover and utilize existing routes in their vicinity, while the drawing capability enables users to manually create custom routes by plotting their own paths. Regardless of the generation method chosen (random, custom, nearby, or manually drawn), users can further edit any route by adding parts, removing sections, or redrawing portions to better fit their preferences.

The exercise plan creating module 110 can enable users to perform free runs where they can begin activity without a predetermined route, with the system collecting and tracking their data as they proceed. Users can draw workout and race courses directly on maps, with the system automatically calculating distance and other relevant metrics in real-time. The drawing functionality can include the ability to undo or erase specific portions, create multiple overlapping laps or disconnected line segments, and save each drawn segment as a separate layer. Users can add unlimited additional layers and duplicate existing laps or line segments as separate layers, which can be inserted at any point in the sequence rather than just consecutively. The system can display both individual layer distances and total route distance, automatically updating these measurements as layers are added, duplicated, or deleted. During navigation, users can view their icon's position relative to the drawn path across multiple map formats including 2D, 3D, elevation, and satellite views. This feature can be particularly useful for routes through unmarked areas like grass, as users can ensure they stay on their intended path. The system can provide real-time distance calculations for accuracy, especially important for races, and can alert users through vibrations if they stray from their drawn route.

The exercise plan creating module 110 can provide split analysis for completed routes. Users can compare actual achieved splits against calculated target splits for both races and workouts. The exercise plan creating module 110 can enable manual split entry and editing after route completion. For repetitive workouts or races, users can multiply a single drawn route segment-for example, creating a 400-meter loop and specifying the desired number of repetitions. The exercise plan creating module 110 can enable comprehensive goal setting for routes. Users can set various types of goals including goal time for the overall route, goal pace for each mile/kilometer/meter, average pace goals for the whole route, specific pace goals for particular miles/kilometers/meters, or goals based on the personal record of the user for that total distance. The goals can be added to main routes, warmups, cooldowns, and free runs. The module provides flexibility in goal timing-users can add goals after selecting a route or before beginning free runs and free workouts. Goal setting remains optional for all route types.

The exercise plan creating module 110 can provide comprehensive warmup and cooldown functionality. Users can choose to add a warmup, cooldown, or both to their routes, cither integrated with the main route or as separate segments. For integrated warmups/cooldowns, users set the desired distances before adding them to the route, which automatically adjusts the total route distance. The data for these segments can be saved separately from the main route data. When warmups and cooldowns are integrated with the main route, the warmup and/or cooldown can maintain similar or identical route attributes based on either user criteria or predetermined criteria from nearby routes. The system 100 can automatically transition between segments-when a warmup is completed, the system 100 can transition to the main route, and after the main route is completed, the system 100 can transition to the cooldown. Users can build separate warmups and cooldowns independently using the same route building process as main routes, find nearby routes, generate random routes, pick start and end points, or perform “free runs” where data is collected without a predetermined route. The separate warmups and cooldowns can be built with distinct route specifications and can overlap the main route or start/end at locations away from the main route.

The exercise plan creating module 110 can enable flexible data collection and adjustment for all route segments. If a route is not completed as planned-such as warmups being cut short, lengthened, or added during the run-data continues to be collected. Users can adjust segment distances and designations after completion, with the system 100 automatically readjusting statistics accordingly. For example, users can designate certain miles as warmup, main route, or cooldown, and save these changes. The exercise plan creating module 110 provides comprehensive route preview capabilities for warmups and cooldowns. Separate segments can be previewed individually or alongside main routes using color coordination. Users can modify routes after completion to examine specific distances or exclude certain portions, with data and statistics automatically adjusting to reflect these changes. The original unmodified data remains available for reference.

The system 100 enables comprehensive data comparison and performance analysis through numerical or graphical representations, with the AI module generating recommendations for improvement based on analyzed data, statistics, and performance metrics while considering factors including goals, conditions, route difficulty, past routes, and current fitness level.

When a user starts but does not complete a planned route, run, or workout, the exercise plan creating module 110 continues to collect and save all data for the completed portion. The data can include pace, distance, heart rate, elevation, terrain, and all other tracked metrics for the portion completed. The partial route data can remain accessible through the system interface for later review, analysis, and comparison, ensuring no activity data is lost regardless of completion status. Users can view this partial data alongside complete route data and incorporate it into their overall training analysis.

For continuous workouts, the exercise plan creating module 110 can automatically record split times when preset distances are reached while continuing to collect data for subsequent splits. Users can pause, stop, and continue workouts as needed. The exercise plan creating module 110 can enable viewing actual splits versus calculated splits during and after workouts, with the ability to edit split times. Total workout distance is displayed and updated throughout the session. The exercise plan creating module 110 can enable real-time route modifications during active sessions. Users can alter the routes by making any portions longer or shorter while in progress, with the system continuing to collect all relevant data and statistics for the modified route. After route completion, users can access the modifications through the exercise plan creating module 110 to readjust and customize the collected data. The system 100 maintains both the original route data and the modified route data, allowing users to compare and analyze the differences between their planned and actual routes.

The exercise plan creating module 110 can display comprehensive route information during preview. Users can view route features and descriptions including route conditions, route pictures, area reviews, traffic patterns, terrain and topography breakdowns. The system can show the percentage match between generated routes and requested criteria. Difficult sections (like steep inclines), easy sections (like steady downhills), and neutral sections (like rolling hills) can be color-coded both in preview and during active routes.

The exercise plan creating module 110 can provide quick return routing functionality during active routes. While users are on a route, users can select an option to return to the starting point or a home location. Rather than simply reversing the route already taken, the module can calculate the fastest (e.g., as based on time or distance) possible return path to that starting point. The optimized return routing can help users efficiently navigate back to their starting location when needed.

The exercise plan creating module 110 can enable performance analysis through color-coded indicators showing where users struggled, performed well, or maintained pace during routes. The indicators are based on factors including fitness level, past performances, route difficulty, and conditions. The system 100 generates recommendations for improvement based on analyzed data/stats/analytics/performance.

The safety module 120 can provide comprehensive safety area integration functionality. Users can choose to incorporate various safety locations including bathrooms, police stations, fire stations, and other safety areas into their routes. The safety module 120 enables two primary ways of utilizing the safety locations. First, users can plan routes to specifically include or stay near the safety areas during the initial route generation. Second, while actively on a route, users can locate and get directions to the closest safety locations as needed. The system 100 provides navigation assistance to guide users to their chosen safety location when requested during an active route.

The system 100 can provide comprehensive emergency contact functionality through the alert module 124. Users can add phone numbers to an emergency contact list or synchronize existing contacts directly into the system. When enabled by the user, the emergency contacts can access the live location of the user during routes. Additionally, the system 100 can be configured to automatically alert emergency contacts when users start or finish routes. The geolocation module 116 can work in conjunction with the safety module 120 to track user location and enable sharing of location data with emergency contacts and services when necessary. The system 100 can maintain location tracking functionality even when service connectivity is limited.

The alert module 124 can provide comprehensive motion detection and alert functionality during routes. Users can manually pause or stop the routes at any time, which halts the time tracking and other statistical measurements until the user either resumes movement or manually hits the continue/unpause/resume button. When a user stops for longer than a first predetermined period, the system 100 can initiate a multi-stage alert process. Initial alerts can be delivered through one or more channels including vibration, sound, and/or messages. If no response is received within a second predetermined period, a second alert is sent. If the second alert receives no response within another of the second predetermined periods, a final alert is issued. Should the user fail to respond to the final alert within another of the second predetermined period, the system 100 can automatically notify one or both of a designated emergency contact and emergency services, sharing a current location of the user.

Users can customize or disable the alert module 124 functionality through various settings. Customization options include selecting alert types (c.g., vibration, sound, messages), adjusting the duration between alerts, specifying which emergency contacts to notify, and setting the number of messages to send before emergency notification. The settings allow users to tailor the safety feature for specific preferences while maintaining the protective function.

The team module 122 can enable comprehensive team and club functionality through a structured registration system that requires careful setup and management. Organizations can first be registered by an individual administrator who creates the organization name and related information. During the registration process, organizations can be designated as either public or private, with different membership requirements established for each type. For private organizations, users can be limited to receive and accept invitations or submit join requests that require administrator approval. School team membership can require an additional layer of verification, such as through a specific school email address. Public organizations can allow immediate joining, though administrators can retain the ability to remove members who may need to request rejoining.

The system 100 can implement a color-coded visual system to display real-time status indicators showing which team members are injured, registered for specific meets, at risk of overtraining based on collected performance data, and tracking daily running completion status. The system 100 can display green indicators for members who have completed their daily runs and red indicators for those who have not. The status tracking can be viewed for any specific date or date range, allowing coaches and administrators to monitor historical participation and completion rates. The system 100 can track and display member status information including injuries, meet attendance, overtraining indicators, and academic year for students. The team module 122′ can provide organization-wide communication and status tracking. Administrators can create and send announcements to specific organizations, multiple organizations, or select individuals. The system 100′ can track completion status for daily training regimes and tasks assigned through coach templates, allowing coaches to monitor progress and verify whether members completed their assigned activities. Performance data can be analyzed and categorized by customizable time periods, including seasons, years, or user-defined date ranges.

The team module 122 can enable workout customization specifically through a coach calculator feature that allows calculation of workout splits and times for specific distances. Users can calculate splits for teams, groups or individuals based on collected performance data. The coach calculator can determine split times based on recent race times, fastest times, personal records, or custom goals. Users can specify the number of repetitions and customize progression rates for splits. For calculating splits, users first select specific individuals or groups and input either a specific time or set a custom time as the base for split calculations. The system can then allow users to specify split distances and target paces for the workout. Based on these inputs, the system can calculate precise split times for each repetition. Users can specify the number of repetitions desired for the workout, and the system can display calculated split times for all selected individuals or groups. The calculated splits can be edited as needed and shared with team members or other coaches. The split calculations can factor in individual performance metrics to provide personalized target times while maintaining group coordination. Users can customize progression rates through options like steady pace (maintaining consistent splits), progressive pace (splits getting faster or slower), alternating pace (alternating between fast and slow splits), or custom combinations. These progression adjustments can be based on either time increments (e.g., 2 seconds faster per repetition) or percentage changes (e.g., 10% faster per repetition).

The team module 122 can enable coaches to create templates to group team members based on similar abilities and times. These templates can allow for predetermined times, splits, workouts, rest periods, and mileage goals, with both default options and full customization capabilities based on coach preferences and team needs. From the templates, coaches can create daily regimes for team members that specify activities to complete. The system 100 can track completion status, allowing coaches to monitor progress and verify whether members completed their assigned tasks. For continuous workouts, the exercise plan creating module 110 can automatically record split times when preset distances are reached while continuing to collect data for subsequent splits. The workout calculator functionality within the team module 122 can provide default settings for common workout types while enabling coaches to fully customize parameters like split distances, paces, progressions and rest periods based on their specific training methodologies and athlete needs.

The system can include race simulation capabilities that provide comprehensive predictive analysis for teams and organizations. The system 100 can generate race simulations by analyzing historical performance data and improvement trends throughout a season. Teams can input data for each runner, including recent race times, training metrics, and seasonal progression rates, to simulate expected performance on specific courses. The simulation factors in course-specific elements like terrain, elevation, and historical course data to generate accurate predictions. Teams can also run virtual race simulations against other teams by inputting competitor times, standings, and publicly available performance data.

The exercise plan creating module 110 can enable comprehensive rest time tracking and split editing capabilities during workouts. The system 100 can display total accumulated rest time between repetitions, allowing users to monitor recovery periods during interval training. Users can edit or remove individual splits during or after the workout when circumstances affect performance, such as falling or other disruptions that may impact split accuracy. When a split is removed, the system 100 can automatically adjust the sequence of remaining splits to maintain continuity. The modifications can be synchronized in real-time with team data, ensuring that coaches and team members have immediate access to updated performance metrics. The system 100 can maintain both the original and modified split data, allowing users to reference both versions while ensuring team statistics reflect the most current edited information. Users can log splits manually by filling in or selecting workout split distance, fill out times for each split completed, and choose the date. The system can enable manual split entry and editing after route completion. For repetitive workouts or races, users can multiply a single drawn route segment-for example, creating a 400-meter loop and specifying the desired number of repetitions.

The team module 122 can implement data analysis and improvement tracking across multiple performance metrics. The system 100 can track race times and paces for standard distances by comparing cach new race against previous performances, analyze workout split times to evaluate the progression of interval speeds over time, monitor average pace per mile or kilometer for training runs, evaluate total weekly and monthly mileage volume metrics or custom dates and ranges, analyze heart rate data trends during similar workouts, and assess recovery metrics between workouts. Users can compare specific or average performance data between selected individuals and their associated organizations, with the ability to select two or more individuals to compare against team averages or specific performance data points. The system can implement a color-coded arrow system that shows a green upward arrow whenever an athlete's time improves compared to their previous performance, while displaying a red downward arrow when performance shows decline. Users can analyze progression throughout seasons, viewing improvements or declines for both individual runners and whole teams across entire seasons or custom date ranges.

An organization-wide communication feature of the team module 122 can allow administrators to create and send announcements to specific organizations, multiple organizations, or select individuals. The system 100 can maintain comprehensive tracking of member status information including injuries, meet attendance, overtraining indicators, and academic year for students. The information can be broken down by season, year, or custom dates, allowing for detailed analysis of team and individual progression over time.

The system 100 can implement detailed improvement tracking across multiple specific performance metrics, which can include tracking race times and paces for standard distances by comparing each new race against previous performances, analyzing workout split times to evaluate the progression of interval speeds over time, monitoring average pace per mile or kilometer for training runs, evaluating total weekly and monthly mileage volume metrics, analyzing heart rate data trends during similar workouts, and/or assessing recovery metrics between workouts. Users can compare specific or average performance data between selected individuals and their associated organizations, with the ability to select two or more individuals to compare against team averages or specific performance data points.

The system 100 can implement comprehensive performance tracking through detailed graphical visualizations of collected data. The display generating module 112 can present performance metrics through multiple graph types that enable thorough analysis of user activities. For individual users, the system can generate pace graphs comparing actual performance against goals, elevation profiles showing route topography changes, and terrain distribution charts breaking down surface types by percentage. The system can display heart rate tracking data for each mile/kilometer segment, calorie burn rates throughout the route, and comprehensive performance indicators using color-coding to highlight areas where users struggled (red), excelled (green), maintained pace (gray), or slowed down (yellow).

For synced activities between multiple users, the display generating module 112 can present comparative data either through side-by-side graphs or overlapped visualizations for direct comparison. The system can generate specialized graphs for different activity types, including RPM tracking for cyclists and step count analysis for walkers, hikers, and runners. Dedicated graphical displays can also track warmup and cooldown segments separately, showing detailed metrics like pace, heart rate, and elevation changes specific to these portions of the workout.

The exercise plan creating module 110 can enable users to analyze performance data across multiple completed activities by connecting separate runs and viewing their statistics cither individually or in combination. The system maintains both original and modified versions of performance data, allowing users to examine their progress through various analytical perspectives. These comprehensive visualization capabilities can help users and coaches track improvement across multiple performance metrics, including race times, workout splits, average paces, and recovery patterns.

The display generating module 112 can provide comprehensive performance analysis through detailed data visualizations and metrics tracking. The system can analyze individual performance based on route variables, providing ratings of poor, average, or great while breaking down performance for each mile/kilometer to show where users struggled, excelled, picked up pace, or slowed down. These analytics can be enhanced with motivational quotes integrated alongside performance data to encourage users during their activities.

For synchronized activities, the display generating module 112 can enable detailed partner workout comparisons through side-by-side data presentation. The system can track and compare synchronized weather conditions, temperature data, and goal achievement metrics between partners. Terrain analysis can be broken down by percentage for each mile/kilometer segment, providing detailed surface composition metrics for the entire route.

The exercise plan creating module 110 can implement specialized analytics for different workout components and activity types. Warmup and cooldown segments can be tracked separately with dedicated pace, heart rate, and elevation graphs. Activity-specific metrics can be monitored, such as RPMs for cyclists and step counts for walkers/runners, with customizable date ranges enabling performance comparisons across different time periods.

The display generating module 112 can enhance route visualization through color-coded sections that indicate difficulty levels and performance metrics. The system can generate performance comparison charts that evaluate user achievement against variables including conditions, goals, route difficulty, and current fitness level. Based on this analyzed data, the system can provide detailed recommendations for improvement, including suggested training adjustments and performance optimization strategies. The system can track time-based metrics through specialized graphs, including comparisons to personal record goals and detailed mile/kilometer breakdowns. Performance data can be visualized through multiple formats, allowing users to analyze their progress through various analytical perspectives while maintaining access to both raw data and graphical representations.

The team module 122 can enable comprehensive race simulation capabilities for teams and organizations. The system 100 can generate predictive race simulations by analyzing historical performance data and improvement trends throughout a season. Teams can input their runners data, including recent race times, training metrics, and/or seasonal progression rates, to simulate expected performance on specific courses. The simulation can factor in course-specific elements like terrain, elevation, and historical course data to generate accurate predictions.

The system 100 can enable teams to run virtual race simulations against other teams or organizations. Teams can input competitor times, standings, and/or publicly available performance data to simulate head-to-head competitions. The simulations can incorporate multiple data points including seasonal best times, average performances, and improvement trajectories. Teams can also simulate races against historical data, allowing comparison against previous years performances on the same courses.

Coaches can create templates to group team members based on similar abilities and times. The templates can allow for predetermined times, splits, workouts, rest, mileage, etc. From the templates, coaches can create daily regimes for team members, which can be a list of activities that team members must complete. Coaches can indicate when each part is completed and the coach can see progress and if members did or did not complete what was asked of them.

For individual athletes, the system 100 can generate personalized race simulations based on their training data and performance progression. The simulation can analyze one or more factors including recent workout times, recovery metrics, and seasonal improvement rates to predict potential race outcomes. Coaches can use the individual simulations to adjust training plans and race strategies based on predicted performances. The simulation functionality can provide detailed analytics including predicted finishing times, projected splits, expected placing within the field, and confidence intervals for the predictions. Teams can view the simulations through various visualization options including course maps, split breakdowns, and head-to-head comparisons. The system can update simulation predictions in real-time as new performance data is collected throughout the season.

The system 100 can enable users to create and customize avatars through an avatar system module. Users can create digital representations that they can interact with and use to track various types of information. The avatars can be customized with team singlets/uniforms displaying logos and colors, shoes, and other custom features. Users can add their own voice to the avatar. The system can track nutrition by “feeding” the avatar and monitoring hydration levels, monitor shoe tread life by tracking mileage on shoes added to the avatar with replacement alerts, and implement sleep tracking through sheep corresponding to hours of sleep jumping over the user's avatar. The system can alert users about overtraining, nutrition needs, and provide exercise reminders. The team module 122 can enable coaches to record custom voice content that can be added to avatars and used for team communications. Through Al integration, coaches' recorded voices can be used to generate customized motivational phrases, workout instructions, and announcements that can be delivered through the avatar system. The voice recordings can be synchronized across team members' devices, allowing coaches to provide consistent messaging and motivation through their personalized avatar voices.

The avatar system can enable interactive features during routes and workouts, allowing users to race their avatars which appear on routes during active sessions. For synced runs done separately or at different times, the avatars can perform race simulations against each other based on completed routes, with multiple avatars visible on synced routes. The avatars can perform dance moves and collect items while running routes with users. Each avatar can have a customizable “walk-up song” that plays when clicking on avatars, during loading screens, countdown sequences, route building processes, and before challenges or races. The avatar system can include mini-games that users can play with their avatars during offline periods, while waiting, or during download screens to maintain engagement with the system.

The display generating module 112 can include achievement tracking through a hall/wall of fame displaying accomplishments, best times, and various totals including mileage, years of running, wins, and teams. Users can collect badges, medals, and awards that can be displayed on or with their avatar when reaching certain milestones or goals. Users can view each other's hall/wall of fame and play mini-games with their avatars during offline periods, while waiting, or during download screens to maintain engagement with the system. The mini-games can include multiple avatars if desired or synced.

The system 100 can maintain comprehensive data collection, storage, and analysis capabilities. When a user starts but does not complete a planned route, run, or workout, the exercise plan creating module 110 continues to collect and save all data for the portion that was completed. Users can track and collect extensive route statistics including total distance, average pace, pace per mile/kilometer, average MPH/KPH, heart rate data, temperature, weather conditions, elevation data, terrain percentages, calories burned, performance ratings, body temperature, and sugar levels. This data can be viewed as raw data or in graphical representations that include comparisons to prior activities. The partial route data remains accessible through the system interface for later review, analysis, and comparison, ensuring no activity data is lost even if the full planned distance is not achieved.

Users can connect multiple completed runs, with the ability to view their data and statistics either together or separately in the databases 107. Data and statistics collected from free runs or free workouts can be added to completed built/generated routes and their associated data. When users perform free runs as warmups or cooldowns (where they simply start and go as fast and as far as they want with tracked data), the data can be connected to data from a completed route. The system 100 can provide flexibility in data viewing-merged data and statistics can be examined either separately or in combination, allowing users to analyze performance across connected activities in various ways. The original unaltered data remains available for reference while enabling the combined views and connections.

The system 100 can enable data adjustment and customization after route completion. Users can exclude parts, combine data from multiple activities, add distance that was not recorded, and alter statistics as needed. The original unaltered data remains available for comparison. Users can connect multiple completed runs and view their data either separately or combined. Data from free runs can be added to completed route data.

The system 100 through the data analysis module 158 can facilitate comprehensive data sharing capabilities. Routes, workouts, and completed activities can be shared with specific groups, public users, the community, or specific individuals/friends. When routes are shared for data/stat comparisons, the routes can be run simultaneously or at different times. Multiple users can compare data side-by-side after route completion.

The system 100 can provide performance analysis through numerical or graphical representations. Individual and team statistics can be compared between multiple users. Performance comparisons can consider factors including goals, conditions, route difficulty, past routes, and fitness level. The system can generate recommendations based on analyzed data/stats/analytics/performance, offering suggestions for improvement, rest, strengthening, and general enhancement.

The system 100 can maintain data accessibility even when device connectivity is limited. Once routes are downloaded or transferred to a device, saved details about the route, terrain, and weather remain accessible even without service or satellite connectivity. For routes that are not completed, the system 100 can still collect and save data for the portion that was completed.

The system 100 can provide voice navigation and suggestions during routes. Users can enable voice guidance that provides real-time recommendations like “A large hill is coming up . . . shorten your stride and lean into it!” The suggestions can use either the users custom voice or a predetermined/default voice from a set of available options.

During active routes, the system 100 collects comprehensive real-time data that can be viewed both during and after the activity. For synced users, the system 100 tracks comparative metrics including relative pace, estimated arrival times, heart rates, calories burned, distances, steps, and RPMs. The system 100 monitors environmental conditions including temperature changes and body temperature throughout activities. After completion, users can access detailed analysis including goal achievement metrics and terrain breakdowns.

The system 100 can provide comprehensive audio pacing functionality via the audio module 126 that can be integrated with music and user playlists. The audio pacing feature uses audio beats and rhythms to help guide user pace and can be enabled alongside music and playlist songs as optional features that users can control as desired. The audio pacing features can be added after routes are chosen and can also be incorporated into free runs and free workouts.

The audio module 126 can implement audio pacing through multiple modes. In goal-matching mode, the audio beat matches the pace of the users set goal. If the user falls behind the target pace, the beat automatically speeds up until the user returns to the desired pace, while maintaining the current speed when the user is ahead of their goal. The goal-matching functionality can be utilized during free runs as well.

In dynamic pace mode, the audio module 126 can adjust the audio beat in real-time to match the user's current pace as they speed up, slow down, or maintain a steady pace. The dynamic pace mode can also be used during free runs, providing real-time audio feedback that corresponds to the actual movement of the user. The exercise plan creating module 110 also offers fixed tempo mode, where the audio beat maintains the same consistent tempo throughout the entire route regardless of the pace of the user. Like the other audio pacing modes, the fixed tempo feature can be utilized during free runs.

As the display 112, the system 100 can provide comprehensive smart device integration through wearable technology like smart glasses that can extend core functionality beyond traditional mobile devices. The smart glasses integration can enable users to experience routes through augmented reality displays, providing an immersive visualization of terrain, paths, and navigation elements. Users can access all standard system features through the smart glasses interface, including performance data monitoring, motivational content delivery, directional guidance, and audio features like smart beat and music integration.

The smart glasses can enable voice command functionality for hands-free system control, allowing users to manage workouts, initiate free runs, and track split times through voice interactions. Safety features can be accessed through the smart glasses interface, with users able to locate and receive directions to nearby safety areas like restrooms, police stations, and first aid stations.

Avatar integration through smart glasses can provide enhanced visualization capabilities, allowing users to see their customized avatars and other users' avatars rendered within their field of view during routes. The system can display avatars performing activities like collecting virtual items and achievements while running, adding an interactive element to the exercise experience. For synced runs performed separately or at different times, the smart glasses can render avatar race simulations based on completed routes, with multiple avatars visible simultaneously.

The smart glasses integration can also incorporate immersive mini-game elements that appear in the user's surrounding environment through augmented reality. These games can be accessed during offline periods, while waiting, or during system loading screens to maintain user engagement. The comprehensive smart glasses functionality can maintain synchronization with other devices, ensuring consistent access to route data, performance metrics, and interactive features across the user's connected devices.

The system 100 can provide comprehensive recommendations based on analyzed performance data through the artificial intelligence module 128. When users struggle with hills, the system can recommend targeted hill training workouts and specific muscle strengthening exercises to improve performance on inclines. The AI module can analyze stride patterns to suggest optimal stride length adjustments at specific points along routes, while also providing recommendations for improving lung capacity through specialized breathing exercises and altitude training protocols.

Based on performance analysis, the system can deliver customized workout recommendations focusing on both fast and slow twitch muscle fiber development. For users who struggle with maintaining pace through their entire route, the system can provide endurance and speed training tips along with suggested pacing strategies-such as starting more conservatively or implementing strategic pace increases. The AI module can generate nutrition guidance including specific calorie intake recommendations and hydration strategies based on environmental conditions and temperature data.

The system can provide recovery optimization recommendations including suggested ice bath protocols and specific stretching routines when analysis indicates potential overtraining risks. Goal adjustments can be recommended based on performance trends, with the system suggesting either more attainable targets or more challenging objectives depending on user progress. The AI module can analyze route difficulty and terrain composition to recommend modifications that optimize training benefit while reducing injury risk.

Through comprehensive performance analysis, the system can identify specific points during routes where users struggled or excelled, providing targeted training suggestions while highlighting successful strategies to maintain. The AI recommendations can include optimal timing for breaks and rest periods based on collected performance data. The system can also suggest alternative routes that align with training goals while accounting for current fitness levels and performance capabilities. These personalized recommendations are continuously refined as the system collects and analyzes additional performance data over time.

The system 100 can provide specialized tracking and analytics for cyclists through dedicated biking metrics. For bikers, the system can track total RPMs, RPMs for each mile/kilometer segment, and average RPM per minute throughout routes. The display generating module 112 can generate specialized RPM graphs showing detailed rotation metrics across route segments, with the ability to compare this data between synced users. Cyclists can view their RPM performance through various graphical representations, including mile-by-mile breakdowns and overall route averages.

For hikers and walkers, the system 100 can implement dedicated step tracking functionality that monitors total steps, steps per mile/kilometer, and average steps per minute throughout routes. The display generating module 112 can present step count data through specialized graphs showing detailed metrics for each route segment. When routes are synced between multiple users, the system can display comparative step data either side-by-side or overlapped, allowing hikers to analyze their performance against other users. These specialized metrics can be integrated with the system's comprehensive performance tracking, enabling hikers to view their step data alongside other metrics like pace, elevation changes, and terrain composition.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods may be made within the scope of the present technology, with substantially similar results.

What is claimed is:

1. A system for creating a customized route to travel for a user, comprising:
a mobile device including:
a human interface for inputting user-determined criteria;
a memory storing processor executable instructions;
a processor in communication with the human interface and the memory;
a mobile display for displaying a customized route;
a system in communication with the mobile device through a wide area network, the system including:
an exercise plan creating module configured to:
receive the user-determined criteria;
generate a customized route based on the user-determined criteria;
enable adding a segment to the customized route;
enable removing a segment of the customized route; and
enable redrawing a segment of the customized route;
a display generating module configured to display the customized route.

2. The system of claim 1, wherein the exercise plan creating module is further configured to selectively generate the customized route through random generation, user-specified start and end points, by locating a nearby existing route, or by manual drawing by the user.

3. The system of claim 1, wherein the display generating module is further configured to enable route preview through one of a 2D map, a 3D map, an elevation map, and a satellite map.

4. The system of claim 1, wherein the exercise plan creating module is further configured to selectively enable integration of a warmup segment and/or a cooldown segment with the customized route.

5. The system of claim 1, wherein the exercise plan creating module is further configured to automatically record split times at preset distances during continuous workouts.

6. The system of claim 1, wherein the exercise plan creating module is further configured to collect and store a performance metric for a completed portion of a route even when the route is not completed by the user.

7. The system of claim 1, further comprising an audio module configured to provide adjustable audio.

8. The system of claim 7, wherein the audio module is configured to adjust an audio tempo based on user-specified parameters.

9. The system of claim 7, wherein the audio module is configured to integrate voice navigation as the user travels the route.

10. The system of claim 7, wherein the audio module is configured to match audio beat with a user-set goal pace and automatically adjust tempo when the user falls behind the user-set goal pace.

11. The system of claim 7, wherein the audio module is configured to maintain an audio pacing functionality during free runs without a predetermined route.

12. The system of claim 1, further comprising a team module configured to enable communication between a plurality of mobile devices of a plurality of users, wherein the team module implements organization registration and membership management for coordinating activities between the plurality of mobile devices of the plurality of users.

13. The system of claim 12, wherein the team module is configured to display a real-time status indicator for the plurality of users, the real-time status indicator including an injury status, a meet registration, and/or an overtraining risk.

14. The system of claim 12, wherein the team module is configured to enable split calculations for each user of the plurality of users based on collected performance data.

15. The system of claim 12, wherein the team module is configured to generate a race simulation using historical performance data and a course-specific element.

16. The system of claim 12, wherein the team module is configured to track improvement across multiple performance metrics and enable comparison between each user of the plurality of users.

17. The system of claim 12, wherein the team module is configured to enable creation of a training template for grouping each user of the plurality of users based on similar abilities.

18. The system of claim 12, wherein the team module is configured to enable organization-wide communication and announcements across the plurality of mobile devices.

19. The system of claim 1, further comprising an avatar module configured to create a customizable digital representation for tracking a user metric.

20. The system of claim 1, wherein the exercise plan creating module is further configured to enable designation of a blocked area based on the user-determined criteria.
